(19) United States
(12) Patent Application Publication
(10) Pub. No.: US 2013/0304206 A1
(43) Pub. Date: Nov. 14, 2013

(54) Intraocular Device to Restore Natural Capsular Tension after Cataract Surgery

(71) Applicants: Ioannis Pallikaris, Heraklion (GR); Harilaos Ginis, Heralkion (GR)

(72) Inventors: Ioannis Pallikaris, Heraklion (GR); Harilaos Ginis, Heralkion (GR)

(21) Appl. No.: 13/468,828

(22) Filed: May 10, 2012

(51) Int. Cl. A61F 2/16

(52) U.S. Cl. 623/6.43

(57) ABSTRACT

Provided herein are a devices, ophthalmic lens systems and methods for restoring natural tension and anatomy of a lens capsule post-surgically in an eye of a subject. The device generally comprises an inward tensioning ring-like structure having a shape configured to circumferentially fit within and be anchored to a post-surgical lens capsule of the eye. The device may have one or both of external and internal grooves formed to receive the lens capsule and one or both of an intraoptical lens or a tensioning element. The ophthalmic lens system generally comprises the device and an intraocular lens inserted therein. The anchored device provides tension to an equatorial area of the capsule resulting in a decrease in equatorial diameter.

### BACKGROUND OF THE INVENTION

1. Field of the Invention

The present invention relates generally to the fields of ophthalmology and cataract surgery. More specifically, the present invention relates to a device implanted in the eye during cataract surgery and improves the optical functionality of the eye.

2. Description of the Related Art

The young healthy eye has the ability to adjust its optical power in order to focus sharply on nearby objects. This function, known as accommodation, is performed by the crystalline lens of the eye. The natural crystalline lens lies in a capsule which is is anchored onto the ciliary body through the zonular fibers. When the ciliary muscle is relaxed, the fibers are taught and the crystalline lens is kept in a relatively “flat” (less refractive) shape and the eye is focused at infinity. Upon observation of a near object, the ciliary muscle contracts, reducing the diameter of the ciliary body, the zonular fibers are relaxed and the crystalline lens is released to take its natural (more convex, more refractive) shape suitable for near focus. When an eye becomes presbyopic, the natural lens loses its flexibility (becomes more rigid) and although the zonular fibers are relaxed the lens does not change its shape significantly. This decline of the eye's ability to focus on nearby objects is called presbyopia and affects the vast majority of the population over the age of 50 years.

Besides presbyopia, aging in the human eye is associated with a progressive loss of the transparency of the crystalline lens. When this loss of transparency becomes a limiting factor for visual performance the natural crystalline lens must be extracted and substituded by an artificial intraocular lens. This loss of transparency when clinically significant (cataract), requires surgical removal of the natural lens and implantation of a polymer intra-ocular lens (IOL) of suitable refractive power.

During surgery, the opacified lens is emulsified and aspirated using an ultrasonic probe inserted through a small incision at the periphery of the cornea. A central opening or capsulorhexis is created on the anterior surface of the lens capsule. Typically, the lens capsule remains in the eye after surgery and is utilised for mounting the intra-ocular lens. The refractive power of the implant is properly calculated in order to focus the treated eye, usually for far objects. Intraocular lens are either static (by design) or have very limited ability to change their focus. In order to perform near work, post-cataract surgery patients usually need to use spectacle correction (reading glasses).

Several designs of intra-ocular lens attempt to perform accommodation by incorporating flexing elements. Their working principle relies on various hypothetical assumptions regarding the mechanism of natural accommodation and moreover on the hypothesis that these mechanisms continue to exist after the opening of the lens capsule and the removal of the opacified lens. In brief, it is hypothesised that the (empty) lens capsule has a persistent inward tension that allows its equatorial diameter to close upon contraction of the ciliary muscle. Presumably, this tension is adequate not only to contract the empty capsule but additionally to flex the mounting structures of the intra-ocular lens (haptics) in a manner to move it axially and therefore to change the focus of the eye for near objects.

However, in clinical practice only minimal refractive changes upon accommodative effort are observed. The reason for this small effectiveness is that after the lens is extracted there is no significant inward tension in the lens capsule capable of deforming the haptics of the intra-ocular lens. It seems that the lens capsule as well as the zonular fibers are always slack regardless of the action of the ciliary muscle. Even if some minimal capsular tension during the immediate postoperative interval is present it is decreased over time (and eventually becomes zero) after fibrosis and loss of elasticity of the lens capsule.

There is a recognized need in the art for an improved device implantable in the eye during or immediately after cataract surgery. More specifically, the prior art is deficient in implantable devices capable of restoring inward tension of the peripheral part of the empty lens capsule after lens extraction. The present invention fulfills this long standing need in the art.

### SUMMARY OF THE INVENTION

The present invention is directed to a device for restoring natural tension and anatomy of a lens capsule post-surgically in an eye of a subject. The device comprises an inward tensioning ring-like structure having a shape configured to circumferentially fit within a post-surgical lens capsule of the eye. The present invention is directed to a related device further comprising a mechanical clamp or an adhesive disposed in anchoring the relationship between the ring-like structure and the lens capsule. The present invention is directed to another related device further comprising a first optical element integrated into the ring-like structure and a second element attachable to the ring-like structure after implantation of the structure. The present invention is further directed to another related device further comprising a tensioning element attachable to the ring-like structure after anchoring the structure to the lens capsule.

The present invention is further directed to an implantable opthalmic lens system for a post-surgical eye in a subject. The lens system comprises the device as described herein and an intraocular lens inserted into the tensioning ring-like structure of the device.

The present invention is directed further to a method for restoring natural tension and anatomy of a lens capsule post-surgically in an eye of a subject. The method comprises anchoring the device as described herein circumferentially to an internal surface of the lens capsule of the post-surgical eye. Tension is provided to an equatorial area of the lens capsule via the ring-like structure comprising the device whereby the ring-like structure directs tension inwardly towards the center of the lens capsule such that an equatorial diameter of the lens capsule is decreased, thereby restoring natural tension and anatomy to the lens capsule. The present invention is directed to a related method further comprising attaching an elastic element to the ring-like structure after anchoring the device to the lens capsule where the elastic element provides the inwardly directed tension to the lens capsule. The present invention is directed to another related method further comprising securing an intraocular lens to the ring-like structure.

The present invention is directed further still to a method for restoring natural tension and anatomy of a lens capsule post-surgically in an eye of a subject. The method comprises inserting the ring-like device described herein circumferentially into an internal surface of the lens capsule of the post-surgical eye. A comb-like device comprising a plurality of spokes is fitted into a space around the lens capsule such that the plurality of spokes are disposed proximately to an equatorial area of the lens capsule whereby the natural tension and anatomy of the lens capsule in the eye is restored. The present invention is directed to a related method in which the comb-like device has a natural diameter smaller than that of the ring-like device and the comb-like device is expanded prior to fitting the same into the lens capsular space.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1C depict cross sections of an eye with a natural healthy lens (FIG. 1A), of an eye that performs accommodation in order to focus on a nearby object. (FIG. 1B) and of the natural shape of the lens if it were disconnected from the zonules (FIG. 1C).

FIGS. 2A-2C compares the cross section of the eye depicted in FIG. 1A

(FIG. 2A) to a cross section of a presbyopic eye that performs accommodation in order to focus on a nearby object (FIG. 2B) and depicts the natural shape of the presbyopic lens if it were disconnected from the zonules (FIG. 2C).

FIGS. 3A-3B depict front (FIG. 3A) and side (FIG. 3B) view of an intraocular lens with mounting structures.

FIGS. 4A-4B depict cross sections of an eye after cataract surgery with an implanted intraocular device (FIG. 4A) and of the same eye under accommodative effort (FIG. 4B).

FIGS. 5A-5C depict cross sections of a ring-like device comprising a groove (FIG. 5A), of an eye after cataract surgery with the ring-like device implanted (FIG. 5B) and of the same eye under accommodative effort (FIG. 5C).

FIGS. 6A-6B depict front (FIG. 6A) and side (FIG. 6B) views of a prior art intraocular lens with optical element and haptics.

FIGS. 7A-7B depict cross sections of an eye after cataract surgery with the ring-like device of FIG. 5A anchored with an adhesive (FIG. 7A) and of the same eye under accommodative effort (FIG. 7B).

FIGS. 8A-8D depict a cross section of an eye having an implanted ring with a groove and an intraocular lens (FIG. 8A), a detailed cartoon of the ring with groove (FIG. 8B), a front view of the intraocular lens with symmetrical haptics (FIG. 8C), and a non-circular capsulorhexis on the anterior surface of the capsule (FIG. 8D).

FIGS. 9A-9B depict a cross section of an eye having a different embodiment of an implanted ring with a groove and an intraocular lens (FIG. 9A) and a detailed cartoon of the ring with groove (FIG. 9B).

FIGS. 10A-10C depict a cross section of a device having an external groove with a clip and an internal groove or hook (FIG. 10A), a ring-like elastic band that hooks to the internal groove (FIG. 10B) and a cross section of the device implanted in an eye (FIG. 10C).

FIGS. 11A-11B depict cross sections of a device with external spoke-like arms shaped similarly to a natural lens (FIG. 11A) and of the device fitted into an empty capsule of an eye (FIG. 11B).

FIGS. 12A-12B depict perspective views of a device comprising external spokes (FIGS. 12A and 12D-12E) where the spokes are inserted separately from the ring (FIGS. 12B-12C).

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device, compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “subject” refers to any recipient of the capsular tension restoring devices and/or lens or ophthalmic lens systems described herein.

In one embodiment of the present invention there is provided a device for restoring natural tension and anatomy of a lens capsule post-surgically in an eye of a subject, comprising an inward tensioning ring-like structure having a shape configured to circumferentially fit within a post-surgical lens capsule of the eye.

Further to this embodiment the device comprises a mechanical clamp or an adhesive disposed in anchoring relationship between the ring-like structure and the lens capsule. As a representative example, a mechanical clamp may comprise a plurality of spokes each disposed between a zonular space and an equatorial area of the lens capsule.

In another further embodiment the device comprises a first optical element integrated into the ring-like structure and a second element attachable to the ring-like structure after anchoring of the structure. As a representative example, the first and second elements may comprise an intraocular lens with haptics. In yet another further embodiment the device comprises a tensioning element attachable to the ring-like structure after anchoring the structure to the lens capsule. As a representative example, the tensioning element may be a comb-like device comprising a plurality of spokes disposed thereon.

In all embodiments the ring-like structure further may comprise a pair of internal grooves disposed in opposition on an internal surface of the ring-like structure. Particularly, the grooves may each have a width sized to receive an intraocular lens. The ring-like structure further may comprise a pair of external grooves with clips formed therefrom where the grooves are disposed in opposition on an external surface of the ring-like structure.

In all embodiments the ring-like structure may have a shape formed to circumferentially and continuously contact an inner surface of the lens capsule. In addition the shape of the ring-like structure may be substantially that of a natural lens peripheral shape. Furthermore natural elasticity of the lens capsule may circumferentially anchor the ring-like structure continuously to an internal capsular surface. Further still the ring-like structure may comprise an elastic material, such as silicone, acrylic or other materials used for the production of foldable IOLs or materials effective as drug delivery vehicles.

In another embodiment of the present invention there is provided an implantable opthalmic lens system for a post-surgical eye in a subject, comprising the device, as described supra; and an intraocular lens inserted into the tensioning ring-like structure of the device. In this embodiment the lens system may comprise at least one element axially movable with a change in an equatorial diameter of the ring-like structure.

In yet another embodiment of the present invention there is provided a method for restoring natural tension and anatomy of a lens capsule post-surgically in an eye of a subject, comprising the steps of anchoring the device, as described supra, circumferentially to an internal surface of the lens capsule of the post-surgical eye; and providing tension to an equatorial area of the lens capsule via the ring-like structure comprising the device whereby the ring-like structure directs tension inwardly towards the center of the lens capsule such that an equatorial diameter of the lens capsule is decreased, thereby restoring natural tension and anatomy to the lens capsule.

Further to this embodiment, after anchoring the device, the method comprises, attaching an elastic element to the ring-like structure, where the elastic element provides the inwardly directed tension to the lens capsule. In another further embodiment the method comprises securing an intraocular lens to the ring-like structure. In this further embodiment the secured intraocular lens may provide the inwardly directed tension to the lens capsule.

In all embodiments the shape of the ring-like structure itself may provide the inwardly directed tension upon anchoring of the device to the lens capsule. In all embodiments the post-surgical lens capsule may comprise a non-circular capsulorhexis on an anterior surface thereof. In addition the circumferential anchoring may comprise a continuous contact between the device and the internal surface of the lens capsule whereby the continuous contact blocks migration of epithelial lens cells to the posterior capsule.

In yet another embodiment of the present invention, there is provided a method for restoring natural tension and anatomy of a lens capsule post-surgically in an eye of a subject, comprising the steps of inserting the ring-like device, as described supra, circumferentially into an internal surface of the lens capsule of the post-surgical eye; and fitting a comb-like device comprising a plurality of spokes into a space around the lens capsule, said plurality of spokes disposed proximately to an equatorial area of the lens capsule whereby the natural tension and anatomy of the lens capsule in the eye is restored.

Further to this embodiment the comb-like device has a natural diameter smaller than that of the ring-like device where the method comprises the step of expanding the comb-like device prior to fitting the same into the lens capsular space. In both embodiments the spokes may be positioned on the comb-like device in a configuration such that the spokes pass between zonules of the eye as the device is fitted into the lens capsular space. Also, in both embodiments the post-surgical lens capsule may comprise a non-circular capsulorhexis on an anterior surface thereof.

Provided herein are devices, systems and methods for restoring natural capsular tension and anatomy postsurgically in the lens capsule of an eye. The tensioning device is anchorable in the lens capsule of the eye after lens extraction, such as during cataract surgery. The tensioning device comprises a ring-like structure that anchors to the peripheral part of the internal capsular surface or is anchored by the natural capsular structure of the lens capsule. The ring-like structure may comprise an elastic material and/or may be a material effective for delivery of a drug, pharmaceutical or other therapeutic compound as is known in the art. For example, the ring-like structure may comprise a plastic, silicon, acrylic, or other material useful for the production of a foldable intraocular lens. The anchored device inwardly directs tension to the capsule. This tension results in a decrease in the equatorial diameter of the capsule upon activation of the ciliary muscle of the eye. The device may be coupled to an appropriate opto-mechanical mechanism to perform accommodation.

The device may be coupled with an opthalmic lens system, such as an intraocular lens with mounting structures. The device is designed, formed or configured to receive an intraocular lens while anchored to the lens capsule. Optionally the intraocular lens may add to or solely provide the inwardly directed tension to reduce the capsular equatorial diameter upon incorporation into the tensioning device. Thus, the present invention also provides a method of restoring capsular tension to a post surgical eye via implantation of the device or ophthalmic lens system into the post surgical lens capsule.

As described below, the invention provides a number of advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

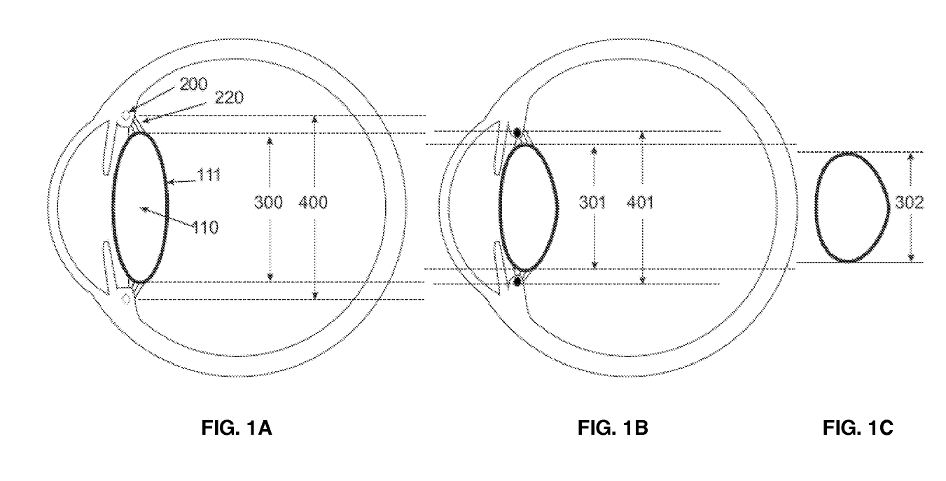

FIGS. 1A, 1B and 1C are cross sectional views of an eye with a natural healthy lens. In FIG. 1A, the crystalline lens 110 is in a capsule 111 and is suspended by the zonular fibers or zonules 220. The ciliary muscle 200 is relaxed and has a maximum diameter 400. The tension of the zonular fibers 220 stretches the lens 110 and capsule 111 to a relatively flat shape having its maximum equatorial diameter at 300. In FIG. 1B the eye has performed an accommodation to focus on a nearby object. The ciliary muscle 200 contracts and constricts its diameter at 401 removing some tension from the zonules 220 and allowing the lens to take a relatively curved shape that has a smaller equatorial diameter at 301. FIG. 1C illustrates the natural shape of the lens 110, if it were disconnected from the zonules 220. Under no equatorial stretch from the zonules the lens 110 has a curved shape and a small equatorial diameter at 302.

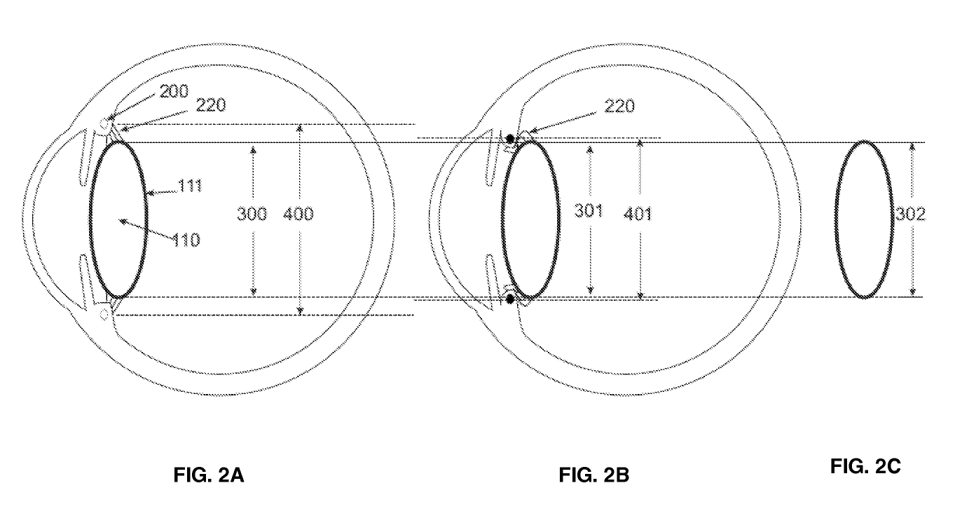

With continued reference to FIGS. 1A-1C, FIGS. 2A-2C are cross sectional views of an eye with a natural presbyopic lens. The eye in FIG. 2A corresponds to that in FIG. 1A in that the crystalline lens 110 is in the capsule 111 and is suspended by the zonular fibers 220. The ciliary muscle 200 is relaxed and has a maximum diameter at 400. The tension of the zonular fibers 220 stretches the lens 110 and capsule 111 to a relatively flat shape having its maximum equatorial diameter at 300. In FIG. 2B, during focus accommodation of the presbyopic eye, the ciliary muscle 200 contracts and constricts its diameter at 401, thereby removing tension from the zonules. Although equatorial tension has been removed from the lens, it does not take a relatively curved shape because it is stiff and does not tend to change its shape. The equatorial diameter of the lens 301 remains almost unchanged as well as its shape. FIG. 2C illustrates the natural shape of the presbyopic lens 110, if it were disconnected from the zonules 220. Under no equatorial stretch from the zonules 220 the lens 110 has a shape and equatorial diameter at 302 very similar to the in situ lens in FIGS. 2A-2B.

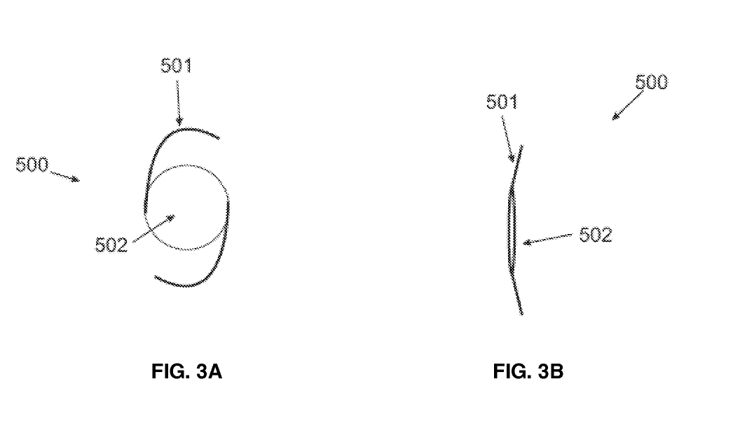

FIGS. 3A and 3B are, respectively, front and side views of an intraocular lens 500. The IOL comprises an optical element 502 and mounting structures, for example, haptics 501.

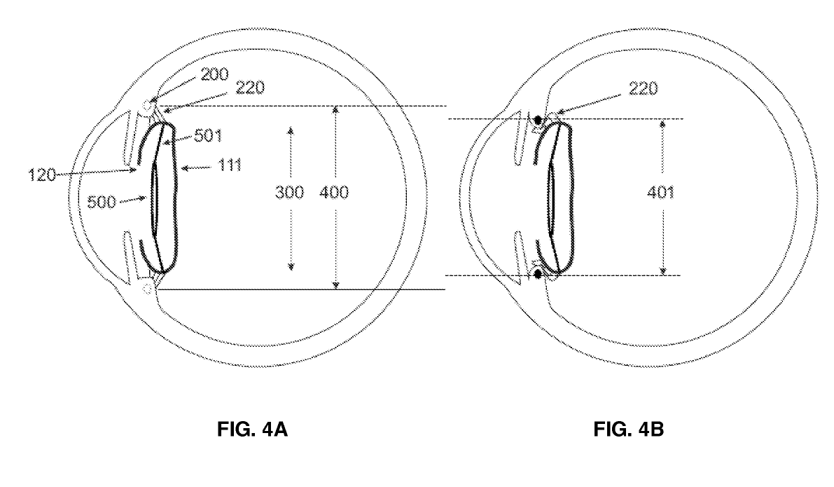

FIGS. 4A and 4B are cross sectional views of an eye after cataract surgery. The natural crystalline lens was extracted through the capsulorhexis opening 120 on the anterior surface of the capsule 111. An intraocular lens 500 is implanted in the empty capsule 111 while its haptics 501 are used to stabilize therein. After lens removal the remaining part of the capsule as well as the zonules 220 are relatively slack. The ciliary muscle 200 is relaxed and has a maximum diameter at 400. The capsule has an equatorial diameter at 300. In FIG. 4B, the eye is under accommodative effort. The ciliary muscle 200 contracts and constricts its diameter at 401 with no effect on the already slack capsule 111 or the intraocular lens inside.

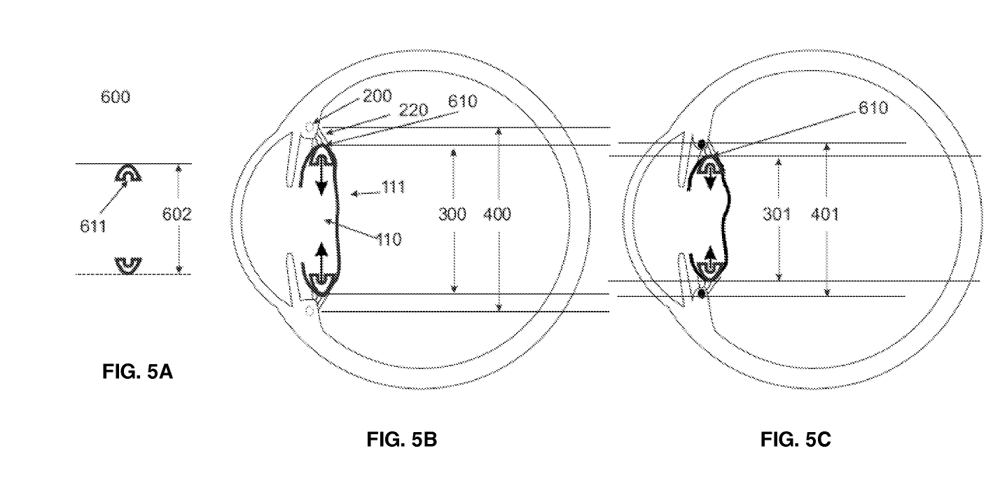

FIGS. 5A, 5B and 5C are cross sectional views of an eye after cataract surgery. The natural crystalline lens 110 has been extracted through a capsulorhexis 120 on the anterior surface of the capsule 111. In FIG. 5A a ring like device 600 that has an equatorial diameter 602 and features a groove 611. In FIG. 5B a ring 600 is stretched equatorially so that its diameter has increased at 300 and has been anchored to the capsule at 610, for example, via an adhesive. The ciliary muscle 200 is relaxed and has a maximum diameter 400. The tension of the zonular fibers 220 stretches the ring 600 and the capsule 111 to its maximum equatorial diameter 300. In FIG. 5C the same eye is under an accommodative effort. The ciliary muscle 200 contracts and constricts its diameter 401 removing some tension from the zonules 220 and allows the ring 600 to take a relatively smaller equatorial diameter 301.

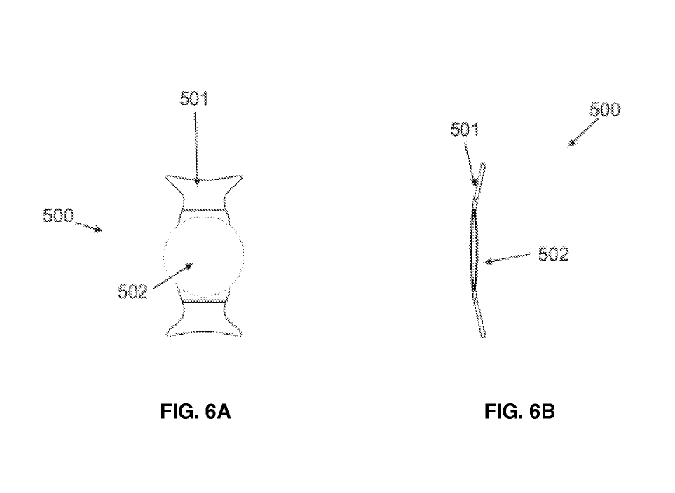

FIGS. 6A and 6B are front and side views of an intraocular lens 500. The intraocular lens comprises an optical element 502 and haptics 501. The haptics are designed so that if mounted in a ring that constricts they will flex pushing the optic 502 along its axis.

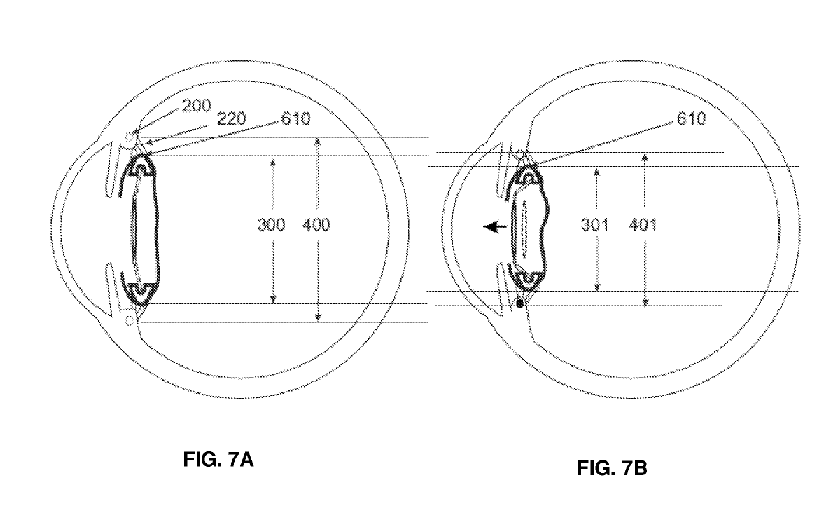

With continued reference to FIGS. 5A-5C, FIGS. 7A and 7B are cross sectional views of an eye after cataract surgery. The natural crystalline lens has been extracted through a capsulorhexis 120 on the anterior surface of the capsule 111. A ring 600 has been stretched equatorially so its diameter has increased at 300 and has been anchored to the capsule at 610, for example, by an adhesive. The ciliary muscle 200 is relaxed and has a maximum diameter 400. The tension of the zonular fibers 220 stretches the ring and the capsule to its maximum equatorial diameter at 300. An intraocular lens 500 comprising flexing haptics 501 is mounted in the ring's groove 611. In FIG. 7B the same eye is under an accommodative effort. The ciliary muscle contracts and constricts its diameter at 401 thereby removing some tension from the zonule fibers and allows the ring 600 to take a relatively smaller equatorial diameter at 301. The ring, by constricting, flexes the haptics 501 of the intraocular lens and therefore the lens moves forward and achieves accommodation.

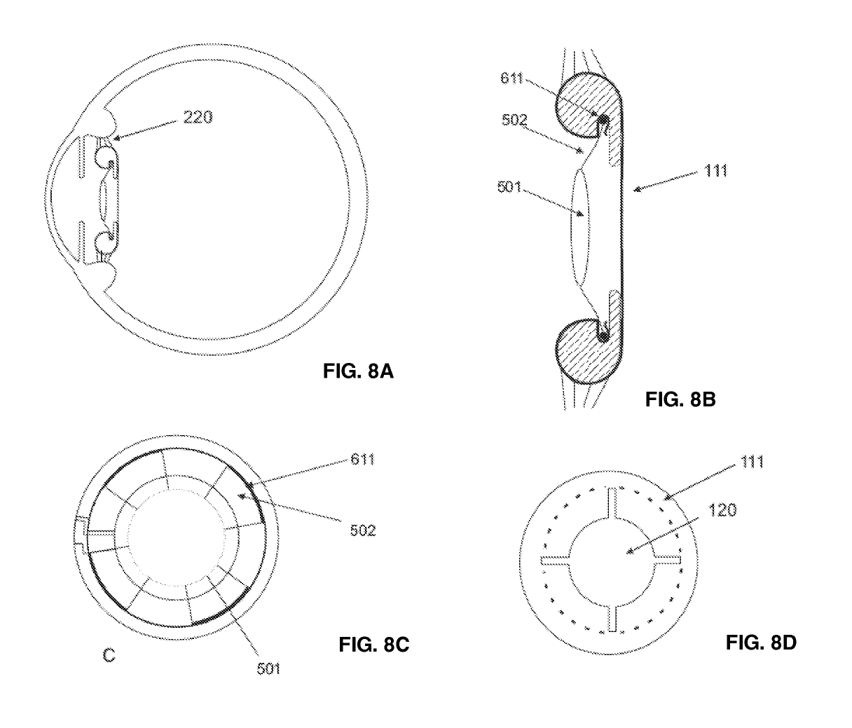

FIGS. 8A, 8B, 8C, and 8D are views of a post surgical eye and an implanted grooved ring and lens system. FIG. 8A shows an eye that has undergone lens extraction and implantation of a ring and an intraocular lens system. FIG. 8B illustrates the details of the shape of the ring with a groove 611 into which the haptics 502 of an intraocular lens 500 can be tightly fitted. The capsule 111 becomes taught as the haptics 502 are pushed into the groove 611. FIG. 8C is a front view of the intraocular lens with a symmetrical placement of haptics 502 that can be used to tighten the capsule 111 in the groove 611 of the ring. FIG. 8D illustrates a non-circular capsulorhexis 120 on the anterior surface of the capsule 111. Such a capsulorhexis may facilitate folding of the capsule 111 and tightening as in FIG. 8B.

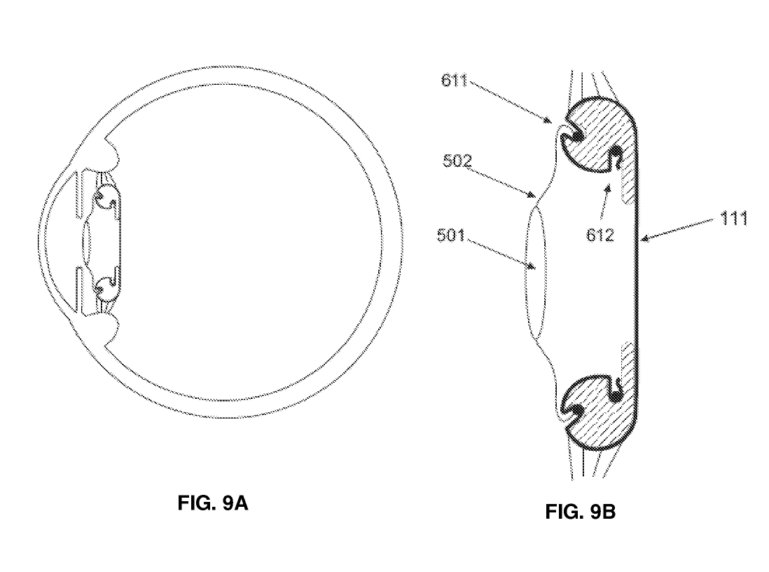

With continued reference to FIG. 8A, FIGS. 9A and 9B are cross sectional views of the post surgical eye and alternate grooved ring device. FIG. 9A illustrates a different embodiment of an implantable ring and an intraocular lens system. FIG. 9B illustrates the details of the shape of the alternate ring featuring a groove 611 into which the haptics 502 of an intraocular lens can be tightly fitted. The capsule 111 becomes taut as the haptics 502 are pushed into the groove 611. These haptics 502 are designed or configured to be flexed in their natural shape and are straightened upon placement into the groove. This creates the inward tension in the system.

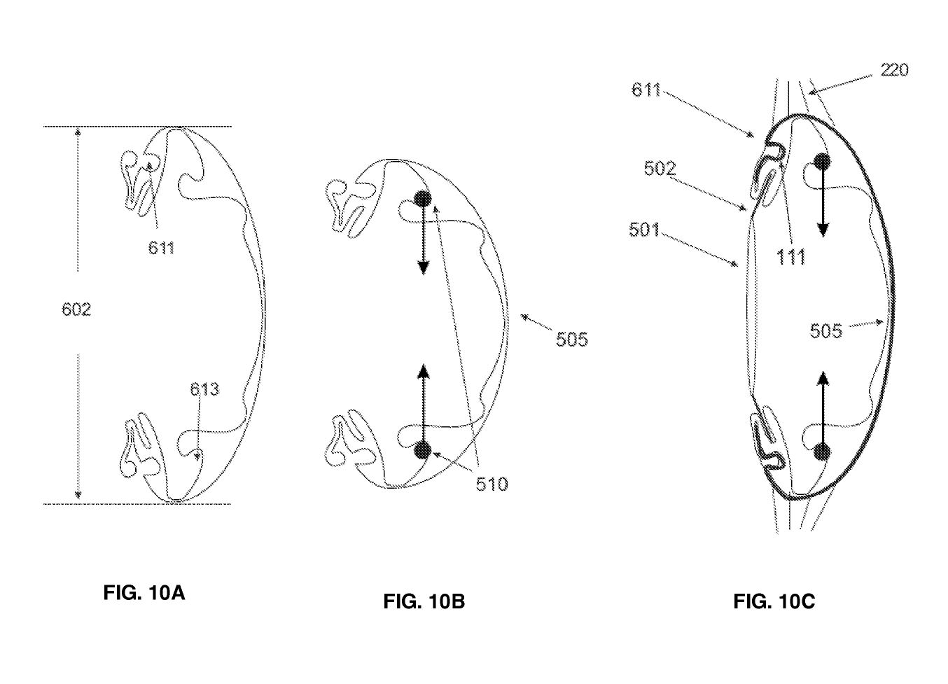

With continued reference to FIG. 1C, FIGS. 10A, 10B and 10C are cross sectional views of a device comprising internal and external grooves. FIG. 10A shows the device with an external groove 611 and associated clip and with an internal groove or hook 613 to which a ring-like elastic band 510 is attached. The initial diameter at 602 of this device and its peripheral equatorial part are comparable or similar to that of the natural lens in the eye. The posterior surface of this device can be transparent and shaped with different internal and external radius of curvature in order to have refractive power and serve as a lens. FIG. 10B illustrates how a ring-like elastic band 510 can be hooked to the internal groove 613 to constrict the device. The device at this state has a natural diameter similar to that of the extracted natural lens at 302 in FIG. 1C. FIG. 10C shows the placement of the device when implanted in the empty capsule 111 of an eye. The capsule is clipped into the grooves 611 when the device is fully expanded and the ring-like elastic band or element 505 is hooked at the designated groove 613 afterwards. Finally, an intraocular lens with an optical element 501 and haptics 502 can be fitted at the anterior side of the device. When the device constricts, the optic 501 moves away from the posterior optic 505 therefore increasing the total dioptric power of the system.

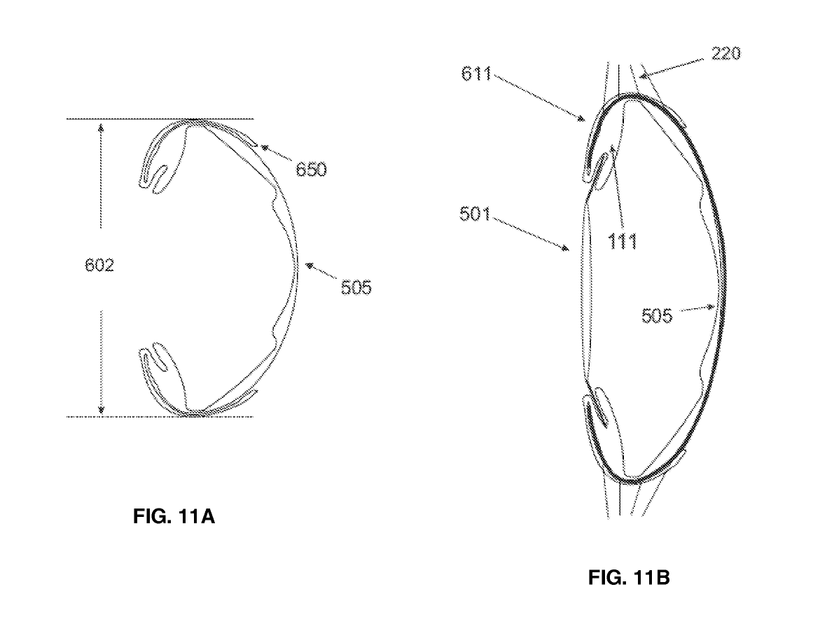

With continued reference to FIG. 1C, FIGS. 11A and 11B are cross sectional views of a device comprising external spokes. FIG. 11A shows the device with 4-8 external spoke-like arms 650 evenly spaced across the meridian which are shaped similarly to the natural lens. The initial diameter at 602 and the shape of the peripheral equatorial part of this device is comparable to that of the natural lens 302. The posterior surface of this device may be transparent and comprises an optical element. FIG. 11B illustrates how this device is fitted in the empty capsule 111 of an eye. The spokes are fitted around the capsule between the zonules 220. Tension from the spokes expands the system to the equatorial diameter of the empty capsule. When the ciliary muscle constricts, the device also constricts under its own tension, the optic 501 is moving away from the posterior optic 505 and therefore increasing the total dioptric power of the system.

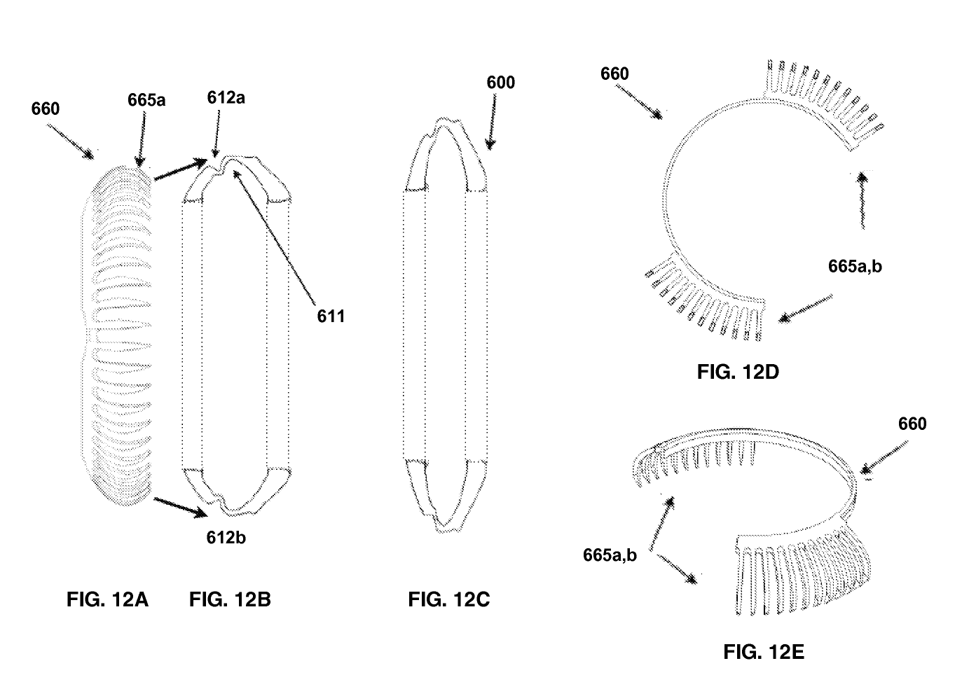

With continued reference to FIGS. 1A, 1B, 1C, and 7B, FIGS. 12A, 12B, 12C, 12D, and 12E are cross-sectional and perspective views of a device comprising external spokes where the spokes are inserted separately from the ring. FIG. 12A shows a device 660 comprising comb-like spokes 665a that can be fitted through the zonules 220 (see FIG. 1A) and provide inward tension against an elastic ring 600 at external grooves 612a,b while at the same time mechanically clamping the capsule (not shown) on the ring. FIG. 12B illustrates the shape of the elastic ring 600 when the forces from the spokes are acting on it. FIG. 12C illustrates the natural shape of the ring 600. FIGS. 12D and 12E are bottom and top views of one configuration of the spokes 665a,b on the device 660.

Tension from the spokes decreases, collapses or restricts the diameter of the system to that of the accommodated natural lens (see FIG. 1B). The elastic ring has a natural diameter similar to that of the un-accommodated natural lens 300 (see FIG. 1C). When the elastic ring is in the capsule and the spokes are fitted around the capsule between the zonules the system remains at almost its maximum diameter 300. When the ciliary muscle constricts, the device also constricts under the tension of the spokes and therefore it can be utilized with an IOL 500 mounted in the ring at groove 611 (see FIG. 7B) to provide accommodation.

The following example(s) are given for the purpose of illustrating various embodiments of the invention and are not meant to limit the present invention in any fashion.

### EXAMPLE 1

Ring Device with Groove: Inward Radial Tensioning

During cataract surgery a capsulorhexis is performed and the cataractous lens is extracted. The capsulorhexis may have additional meridonial openings (FIG. 8D) that will facilitate folding of the empty capsule in the manner described below. The ring-like device that is generally elastic and features an internal groove is inserted in the capsule and the edge of the capsulorhexis is folded (FIG. 8B). The device can be either a complete ring expanding by its elastic properties or it can be less elastic and feature an opening with overlapping edges (FIG. 8C).

An intraocular lens with flexible haptics in inserted into the groove and the tension of the haptics forces the ring to expand and take a wide equatorial diameter. At this condition the ciliary muscle is fully relaxed, the ciliary body has its maximum diameter and the zonules are relaxed. Upon insertion of the lens haptics and folding of the capsulorhexis edge the capsule is stretched and tension in the zonules is restored. At this condition where the haptics are snuggly holding the capsule in the groove the whole system will stay at this maximum diameter as the tension from the zonules will not allow it to close.

After surgery, when the patient fixates at a near target, the ciliary muscle contracts, the tension in the zonules decreases and the device is reducing its diameter forcing the lens to move (axially) forward. This movement results to a change in the total refractive power of the eye and near focus is achieved. When the person fixates to a far object the ciliary muscle will relax and the tension from the zonules will pull the ring/lens system to its large diameter state and the total diotric power of the eye to a lower state, suitable for far vision.

The functionality of the system is based on the appropriate relationship of the radial tensions from the ring, the lens and the zonules. Namely, the ring/lens system is designed to be in a small diameter state at the absence of tension from the zonules. Zonular tension that is obtained by the implantation method and folding of the capsule is keeping it in an expanded state. Here the ring is providing an inward radial tension and the lens is providing an outward tension. The exact diameter of equilibrium is determined by the third tension—provided by the zonules to which the system is anchored. Additionally to the accommodative functionality of this system, the ring has a complete circumferential contact with the inner surface of the peripheral part of the empty capsule therefore preventing the migration of epithelial cells from the remaining part of the anterior to the posterior side of the capsule and therefore is preventing the posterior capsule opacification.

### EXAMPLE 2

Ring Device with Groove: Outward Radial Tensioning

During cataract surgery a capsulorhexis is performed and the cataractous lens is extracted. The capsulorhexis may have additional meridonial openings (FIG. 8D) that will facilitate folding of the empty capsule in the manner described below. A ring-like device that is generally elastic and features an internal and an external groove is inserted in the capsule and the edge of the capsulorhexis is clamped at the external groove (FIG. 9B). An internal ring may be additionally inserted in order to hold the edge of the capsule secured in an internal groove. The device can be either a complete ring expanding by its elastic properties or it can be less elastic and feature an opening with overlapping edges (FIG. 8C).

The tension of the haptics is forcing the ring to decrease in diameter. At this condition the ciliary muscle is fully relaxed, the cliary body has its maximum diameter and the zonules are relaxed. Upon insertion of the lens haptics and clamping of the capsule the capsule is stretched and tension in the zonules is restored. At this condition where the capsule is snuggly held by the lens haptics. The whole system will stay at this maximum diameter as the tension from the zonules will not allow it to close, even though the tension from the lens haptics is tending to decrease its diameter.

After surgery, when the patient fixates at a near target, the ciliary muscle contracts, the tension in the zonules decreases and the device is reducing its diameter forcing the lens to move (axially) forward. This movement results to a change in the total refractive power of the eye and near focus is achieved. When the person fixates to a far object the ciliary muscle will relax and the tension from the zonules will pull the ring/lens system to its large diameter state and the total diotric power of the eye to a lower state, suitable for far vision.

The functionality of the system is based on the appropriate relationship of the radial tensions from the ring, the lens and the zonules. Namely, the ring/lens system is designed to be in a small diameter state at the absence of tension from the zonules. Zonular tension (that is obtained by the implantation method and folding of the capsule is keeping it in an expanded state. The ring is providing an outward radial tension and the lens is providing an inward tension. The exact diameter of equilibrium is determined by the third tension which is provided by the zonules to which the system is anchored. Additionally to the accommodative functionality of this system, the ring has a complete circumferential contact with the inner surface of the peripheral part of the empty capsule therefore preventing the migration of epithelial cells from the remaining part of the anterior to the posterior side of the capsule and therefore is preventing the posterior capsule opacification.

### EXAMPLE 3

Ring Device with External Groove and Clip and Internal Groove: Outward Radial Tensioning

During cataract surgery a capsulorhexis is performed and the cataractous lens is extracted. The capsulorhexis may have additional meridonial openings (FIG. 8D) that will facilitate folding of the empty capsule in the manner described below. A ring-like device that is generally elastic and features a number of internal and external grooves (FIG. 10A) is inserted in the capsule and the edge of the capsulorhexis is clamped at the external groove with special clips that are integral part of the ring (FIG. 10C). An elastic band can be inserted in the ring's internal groove (FIG. 10C). This elastic band will tend to reduce the diameter of the system (FIG. 10B). The device can be either a complete ring expanding by its elastic properties or it can be less elastic and feature an opening with overlapping edges (FIG. 10C). The tension of the elastic band is forcing the ring to decrease in diameter. At this condition the ciliary muscle is fully relaxed, the cliary body has its maximum diameter and the zonules are relaxed.

Upon insertion of the elastic band and clamping of the capsule the capsule is stretched and tension in the zonules is restored. At this condition where the capsule is snuggly held by the ring clips. The whole system will stay at this maximum diameter as the tension from the zonules will not allow it to close, even though the tension from the elastic band is tending to decrease its diameter. An appropriate intraocular lens can be inserted at the anterior side of the ring after the elastic band. The posterior side of the ring can be made to feature an additional optical element.

After surgery, when the patient fixates at a near target, the ciliary muscle contracts, the tension in the zonules decreases and the device is reducing its diameter forcing the implanted lens to move in respect to the posterior lens of the ring. This movement results to a change in the total refractive power of the eye and near focus is achieved. When the person fixates to a far object the ciliary muscle will relax and the tension from the zonules will pull the ring/lens/elastic band system to its large diameter state and the total dioptric power of the eye to a lower state, suitable for far vision.

The functionality of the system is based on the appropriate relationship of the radial tensions from the ring, the lens, the elastic band and the zonules. Namely, the ring/lens/elastic band system is designed to be in a small diameter state at the absence of tension from the zonules. Zonular tension, that is obtained by the implantation method and folding of the capsule, is keeping it in an expanded state. In this embodiment, the ring is providing an outward radial tension, as well as the intraocular lens and the main inward tension is provided by the elastic band. The exact diameter of equilibrium is determined by the third tension—provided by the zonules to which the system is anchored. Additionally to the accommodative functionality of this system, the ring has a complete circumferential contact with the inner surface of the peripheral part of the empty capsule therefore preventing the migration of epithelial cells from the remaining part of the anterior to the posterior side of the capsule and therefore is preventing the posterior capsule opacification.

### EXAMPLE 4

Ring Device with External Spoke-Like Arms: Inward Radial Tensioning

During cataract surgery a capsulorhexis is performed and the cataractous lens is extracted. A generally elastic ring-like device that is generally elastic and features an internal groove is inserted in the capsule. The device features external spoke-like arms that follow the general shape of the equatorial part of the device and pass between the zonules to clamp mechanically the capsule to equatorial area of the device. The device can be either a complete ring expanding by its elastic properties or it can be less elastic and feature an opening with overlapping edges (FIG. 8C).

An intraocular lens with flexible haptics is inserted in the groove. At this condition the ciliary muscle is fully relaxed, the cliary body has its maximum diameter and the zonules are relaxed. Upon insertion of the ring and passing of the spokes between the zonules the tension of the zonules is restored. At this condition the whole system will stay at this maximum diameter as the tension from the zonules will not allow it to close.

After surgery, when the patient fixates at a near target, the ciliary muscle contracts, the tension in the zonules decreases and the device is reducing its diameter forcing the lens to move (axially) forward. This movement results to a change in the total refractive power of the eye and near focus is achieved. When the person fixates to a far object the ciliary muscle will relax and the tension from the zonules will pull the ring/lens system to its large diameter state and the total diotric power of the eye to a lower state, suitable for far vision.

The functionality of the system is based on the appropriate relationship of the radial tensions from the ring, the lens and the zonules. Namely, the ring/lens system is designed to be in a small diameter state at the absence of tension from the zonules. Zonular tension that is obtained by the implantation method and equatorial clamping of the capsule is keeping it in an expanded state. In this embodiment, the ring is providing an inward radial tension and the lens is providing a small outward tension. The exact diameter of equilibrium is determined by the third tension which are provided by the zonules to which the system is anchored. Additionally to the accommodative functionality of this system, the ring has a complete circumferential contact with the inner surface of the peripheral part of the empty capsule therefore preventing the migration of epithelial cells from the remaining part of the anterior to the posterior side of the capsule and therefore is preventing the posterior capsule opacification.

### EXAMPLE 5

Device with External Comb-Like Spokes

During cataract surgery a capsulorhexis is performed and the cataractous lens is extracted. A generally elastic ring device that features an internal groove is inserted in the capsule that fills the empty capsule (FIG. 12A-12C). A comb-like device (FIGS. 12D-12E) having a number of spokes resembling the shape of the peripheral natural lens is inserted. The natural size of this device is smaller than the previously implanted ring. After insertion the device is expanded and hooked around the capsule with its spokes passing between zonules and therefore providing inward tension to the zonules and the elastic ring in the capsule. In the absence of capsular tension the comb-like device would be strong enough to collapse the elastic ring, but at this state of the eye, the zonular tension is keeping the system expanded. The comb-like device may comprise spokes corresponding to all the circumference of the lens equator or cover opposite quadrants (or even smaller sectors) as shown in FIGS. 12D and 12E. In this case two or more of these spoke-like devices can be implanted to provide inward tension across different meridians.

An intraocular lens with flexible haptics is inserted in the groove. At this condition the ciliary muscle is fully relaxed, the ciliary body has its maximum diameter and the zonules are relaxed. Upon insertion of comb-like device between the zonules, the tension of the zonules is restored. At this condition, the whole system will stay at this maximum diameter as the tension from the zonules will not allow it to close.

After surgery, when the patient fixates at a near target, the ciliary muscle contracts, the tension in the zonules decreases and the device is reducing its diameter forcing the lens to move (axially) forward. This movement results to a change in the total refractive power of the eye and near focus is achieved. When the person fixates to a far object the ciliary muscle will relax and the tension from the zonules will pull the ring/lens system to its large diameter state and the total diotric power of the eye to a lower state, suitable for far vision.

The functionality of the system is based on the appropriate relationship of the radial tensions from comb-like component, the ring, the lens and the zonules. Namely, the ring/lens system is designed to be in a large diameter state at the absence of tension from the zonules. Zonular tension (that is obtained by the implantation of the comb-like device and equatorial clamping of the capsule is keeping it in an expanded state. In this embodiment, the ring is providing an outward radial tension, the lens is providing a small outward tension and the comb-like device is providing an inward tension. The exact diameter of equilibrium is determined by the third tension which is provided by the zonules to which the system is anchored. Additionally to the accommodative functionality of this system, the ring has a complete circumferential contact with the inner surface of the peripheral part of the empty capsule therefore preventing the migration of epithelial cells from the remaining part of the anterior to the posterior side of the capsule and therefore is preventing the posterior capsule opacification.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. A device for restoring natural tension and anatomy of a lens capsule post-surgically in an eye of a subject, comprising:
an inward tensioning ring-like structure having a shape configured to circumferentially fit within a post-surgical lens capsule of the eye.

2. The device of claim 1, said ring-like structure further comprising a pair of internal grooves disposed in opposition on an internal surface of the ring-like structure.

3. The device of claim 2, wherein the grooves each have a width sized to receive an intraocular lens.

4. The device of claim 1, said ring-like structure further comprising a pair of external grooves with clips formed therefrom, said grooves disposed in opposition on an external surface of the ring-like structure.

5. The device of claim 1, wherein the ring-like structure has a shape formed to circumferentially and continuously contact an inner surface of the lens capsule.

6. The device of claim 1, wherein the shape of the ring-like structure is substantially that of a natural lens peripheral shape.

7. The device of claim 1, wherein natural elasticity of the lens capsule circumferentially anchors the ring-like structure continuously to an internal capsular surface.

8. The device of claim 1, further comprising:
a mechanical clamp or an adhesive disposed in anchoring relationship between the ring-like structure and the lens capsule.

9. The device of claim 8, wherein the mechanical clamp comprises a plurality of spokes each disposed between a zonular space and an equatorial area of the lens capsule.

10. The device of claim 1, further comprising:
a first optical element integrated into the ring-like structure; and
a second element attachable to the ring-like structure after anchoring of the structure.

11. The device of claim 10, wherein the first and second elements comprise an intraocular lens with haptics.

12. The device of claim 1, further comprising:
a tensioning element attachable to the ring-like structure after anchoring the structure to the lens capsule.

13. The device of claim 12, wherein the tensioning element is a comb-like device comprising a plurality of spokes disposed thereon.

14. The device of claim 1, wherein the ring-like structure comprises an elastic material or a material effective as a drug delivery vehicle.

15. An implantable opthalmic lens system for a post-surgical eye in a subject, comprising:
the device of claim 1; and
an intraocular lens inserted into the tensioning ring-like structure of the device.

16. The lens system of claim 15, comprising at least one element axially movable with a change in an equatorial diameter of the ring-like structure.

17. A method for restoring natural tension and anatomy of a lens capsule post-surgically in an eye of a subject, comprising the steps of:
anchoring the device of claim 1 circumferentially to an internal surface of the lens capsule of the post-surgical eye; and
providing tension to an equatorial area of the lens capsule via the ring-like structure comprising the device whereby the ring-like structure directs tension inwardly towards the center of the lens capsule such that an equatorial diameter of the lens capsule is decreased, thereby restoring natural tension and anatomy to the lens capsule.

18. The method of claim 17, wherein the shape of the ring-like structure itself provides the inwardly directed tension upon anchoring of the device to the lens capsule.

19. The method of claim 17, wherein the post-surgical lens capsule comprises a non-circular capsulorhexis on an anterior surface thereof.

20. The method of claim 17, wherein after anchoring the device, the method further comprises:
attaching an elastic element to the ring-like structure, said elastic element providing the inwardly directed tension to the lens capsule.

21. The method of claim 20, wherein the circumferential anchoring comprises a continuous contact between the device and the internal surface of the lens capsule, said continuous contact blocking migration of epithelial lens cells to the posterior capsule.

22. The method of claim 17, further comprising:
securing an intraocular lens to the ring-like structure.

23. The method of claim 22, wherein the secured intraocular lens provides the inwardly directed tension to the lens capsule.

24. A method for restoring natural tension and anatomy of a lens capsule post-surgically in an eye of a subject, comprising the steps of:
inserting the ring-like device of claim 1 circumferentially into an internal surface of the lens capsule of the post-surgical eye; and
fitting a comb-like device comprising a plurality of spokes into a space around the lens capsule, said plurality of spokes disposed proximately to an equatorial area of the lens capsule whereby the natural tension and anatomy of the lens capsule in the eye is restored.

25. The method of claim 24, wherein the comb-like device has a natural diameter smaller than that of the ring-like device, said method further comprising the step of expanding the comb-like device prior to fitting the same into the lens capsular space.

26. The method of claim 24, wherein the spokes are positioned on the comb-like device in a configuration such that the spokes pass between zonules of the eye as the device is fitted into the lens capsular space.

27. The method of claim 24, wherein the post-surgical lens capsule comprises a non-circular capsulorhexis on an anterior surface thereof.
